(19) United States
(12) Patent Application Publication
(10) Pub. No.: US 2023/0298706 A1
(43) Pub. Date: Sep. 21, 2023

(54) METHODS, MEDIUMS, AND SYSTEMS FOR DETERMINING VARIATION RELATING TO COMPOUND STRUCTURES

(71) Applicant: Waters Technologies Ireland Limited, Dublin 2 (IE)

(72) Inventors: Johannes Vissers, Breda (NL); Keith Richardson, High Peak (GB)

(21) Appl. No.: 18/184,943

(22) Filed: Mar. 16, 2023

Related U.S. Application Data
(60) Provisional application No. 63/320,419, filed on Mar. 16, 2022.

(51) Int. Cl. G16C 20/30; G16C 20/50

(52) U.S. Cl. CPC G16C 20/30; G16C 20/50

(57) ABSTRACT

Exemplary embodiments pertain to methods, mediums, and systems for using molecular properties of chemical compounds to quantify error or variation in collision cross-section predictions. For example, the CCS prediction may determine a location of charge on the compound, and the molecular properties (such as the length normalized residue value or Van der Waals volume of the compound) may be used to assign an error value to the prediction. These error values may be used to build a model of the chemical compound using the error or variance, where the compound is capable of exhibiting more than one value for the CCS value.

### CROSS-REFERENCE TO RELATED APPLICATIONS

This application claims the benefit of and priority to U.S. Provisional Application No. 63/320,419, filed on Mar. 16, 2022, the entire disclosure of which is hereby incorporated by reference herein in its entirety for all purposes.

### BACKGROUND

Mass spectrometry (MS) and liquid chromatography-mass spectrometry (LC-MS) apparatuses are used to analyze a chemical sample to study the identity, mass, or structure of the sample.

Chemical compounds may exhibit structures that can be measured by an LC-MS instrument in the form of collision cross-section values. In some cases, molecular modeling (MM) or machine learning (ML) may also be used to predict CCS values. When compared to measured CCS values, many CCS predictions approximate the error range of experimentally-obtained results when considered as a group. However, individual predictions tend not to be associated with error or variance estimations because these can depend on a number of unknown parameters, such as underrepresentation of a given chemical class or combination of features within the model, variation in the experimental measurement (conformers, ESI and IM-MS conditions, etc.), structural unambiguity, or a combination of any of these parameters.

### BRIEF SUMMARY

The present inventors have discovered that a link exists between CCS prediction error and certain molecular properties of the chemical sample being modeled.

Exemplary embodiments pertain to methods, mediums, and systems for using molecular properties of chemical compounds to quantify error or variation in collision cross-section predictions. For example, the CCS prediction may be associated with a location of charge on the compound that determines the CCS value(s) for the compound, and the molecular properties (such as the length normalized residue value or Van der Waals volume of the compound) may be used to assign an error value to the prediction. These error values may be used to build a model of the chemical compound using the error or variance, where the compound is capable of exhibiting more than one value for the CCS value.

Some embodiments may determine that a given compound is likely to be associated with more than one CCS value, or a CCS prediction value with more variation associated with it (since the structure cannot accommodate the charge). If a compound can exhibit more than one CCS value, since the structure can accommodate a charge at more than one location, this information could be captured/encoded when the model is created, thereby providing the ability to report more than one CCS value. Or, additionally, when charge assignment is challenged, this may be flagged and reported in the output when CCS values are computed (in both ML and MM).

Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an exemplary artificial intelligence/machine learning (AI/ML) system suitable for use with exemplary embodiments.

FIG. 2 depicts an illustrative computer system architecture that may be used to practice exemplary embodiments described herein.

FIG. 3 illustrates an example of a mass spectrometry system according to an exemplary embodiment.

FIG. 4 is a flowchart depicting an exemplary technique in accordance with an embodiment.

### DETAILED DESCRIPTION

### A Note on Data Privacy

Some embodiments described herein make use of training data or metrics that may include information voluntarily provided by one or more users. In such embodiments, data privacy may be protected in a number of ways.

For example, the user may be required to opt in to any data collection before user data is collected or used. The user may also be provided with the opportunity to opt out of any data collection. Before opting in to data collection, the user may be provided with a description of the ways in which the data will be used, how long the data will be retained, and the safeguards that are in place to protect the data from disclosure.

Any information identifying the user from which the data was collected may be purged or disassociated from the data. In the event that any identifying information needs to be retained (e.g., to meet regulatory requirements), the user may be informed of the collection of the identifying information, the uses that will be made of the identifying information, and the amount of time that the identifying information will be retained. Information specifically identifying the user may be removed and may be replaced with, for example, a generic identification number or other non-specific form of identification.

Once collected, the data may be stored in a secure data storage location that includes safeguards to prevent unauthorized access to the data. The data may be stored in an encrypted format. Identifying information and/or non-identifying information may be purged from the data storage after a predetermined period of time.

Although particular privacy protection techniques are described herein for purposes of illustration, one of ordinary skill in the art will recognize that privacy protected in other manners as well. Further details regarding data privacy are discussed below in the section describing network embodiments.

Assuming a user's privacy conditions are met, exemplary embodiments may be deployed in a wide variety of messaging systems, including messaging in a social network or on a mobile device (e.g., through a messaging client application or via short message service), among other possibilities. An overview of exemplary logic and processes for engaging in synchronous video conversation in a messaging system is next provided.

### Exemplary Embodiments

As an aid to understanding, a series of examples will first be presented before detailed descriptions of the underlying implementations are described. It is noted that these examples are intended to be illustrative only and that the present invention is not limited to the embodiments shown.

Reference is now made to the drawings, wherein like reference numerals are used to refer to like elements throughout. In the following description, for purposes of explanation, numerous specific details are set forth in order to provide a thorough understanding thereof. However, the novel embodiments can be practiced without these specific details. In other instances, well known structures and devices are shown in block diagram form in order to facilitate a description thereof. The intention is to cover all modifications, equivalents, and alternatives consistent with the claimed subject matter.

In the Figures and the accompanying description, the designations “a” and “b” and “c” (and similar designators) are intended to be variables representing any positive integer. Thus, for example, if an implementation sets a value for a=5, then a complete set of components 122 illustrated as components 122-1 through 122-a may include components 122-1, 122-2, 122-3, 122-4, and 122-5. The embodiments are not limited in this context.

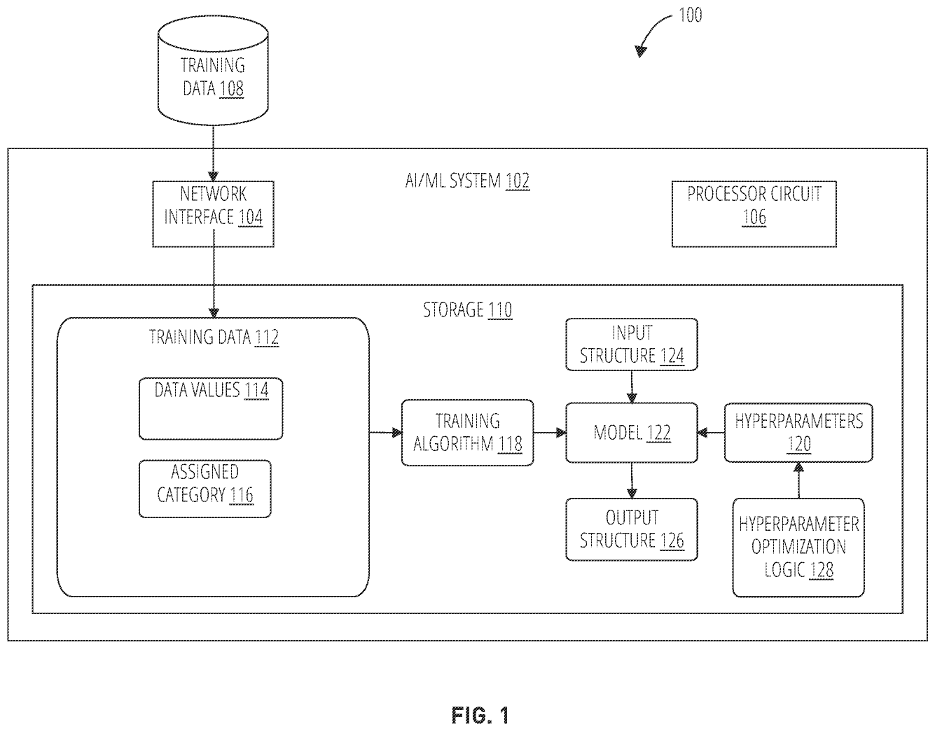

Exemplary embodiments may make use of artificial intelligence/machine learning (AI/ML). FIG. 1 depicts an AI/ML environment 100 suitable for use with exemplary embodiments.

At the outset it is noted that FIG. 1 depicts a particular AI/ML environment 100 and is discussed in connection with particular types of AI/ML architectures. However, other AI/ML systems also exist, and one of ordinary skill in the art will recognize that AI/ML environments other than the one depicted may be implemented using any suitable technology.

The AI/ML environment 100 may include an AI/ML System 102, such as a computing device that applies an AI/ML algorithm to learn relationships between the above-noted protein parameters.

The AI/ML System 102 may make use of training data 108. In some cases, the training data 108 may include pre-existing labeled data from databases, libraries, repositories, etc. The training data 108 may include, for example, rows and/or columns of data values 114. The training data 108 may be collocated with the AI/ML System 102 (e.g., stored in a Storage 110 of the AI/ML System 102), may be remote from the AI/ML System 102 and accessed via a Network Interface 104, or may be a combination of local and remote data. Each unit of training data 108 may be labeled with an assigned category 116 (or multiple assigned categories); for instance, each row and/or column may be labeled with a classification. In some embodiments, the training data may include individual data elements (e.g., not organized into rows or columns) and may be labeled on an individual basis.

As noted above, the AI/ML System 102 may include a Storage 110, which may include a hard drive, solid state storage, and/or random access memory.

The Training Data 112 may be applied to train a model 122. Depending on the particular application, different types of models 122 may be suitable for use. For instance, exemplary embodiments may make use of Bayesian hierarchical models or gradient boosted trees may be particularly well-suited to learning associations the data values 114 and the assigned category 116. In other examples, an deep learning architectures such as a recurrent neural network (RNN). Other types of models 122, or non-model-based systems, may also be well-suited to the tasks described herein, depending on the designers goals, the resources available, the amount of input data available, etc.

Any suitable Training Algorithm 118 may be used to train the model 122. Nonetheless, the example depicted in FIG. 1 may be particularly well-suited to a supervised training algorithm. For a supervised training algorithm, the AI/ML System 102 may apply the data values 114 as input data, to which the resulting assigned category 116 may be mapped to learn associations between the inputs and the labels. In this case, the assigned category 116 may be used as a labels for the data values 114.

The Training Algorithm 118 may be applied using a Processor Circuit 106, which may include suitable hardware processing resources that operate on the logic and structures in the Storage 110. The Training Algorithm 118 and/or the development of the trained model 122 may be at least partially dependent on model Hyperparameters 120; in exemplary embodiments, the model Hyperparameters 120 may be automatically selected based on Hyperparameter Optimization logic 128, which may include any known hyperparameter optimization techniques as appropriate to the model 122 selected and the Training Algorithm 118 to be used.

Optionally, the model 122 may be re-trained over time.

In some embodiments, some of the Training Data 112 may be used to initially train the model 122, and some may be held back as a validation subset. The portion of the Training Data 112 not including the validation subset may be used to train the model 122, whereas the validation subset may be held back and used to test the trained model 122 to verify that the model 122 is able to generalize its predictions to new data.

Once the model 122 is trained, it may be applied (by the Processor Circuit 106) to new input data. The new input data may include unlabeled data stored in a data structure, potentially organized into rows and/or columns. This input to the model 122 may be formatted according to a predefined input structure 124 mirroring the way that the Training Data 112 was provided to the model 122. The model 122 may generate an output structure 126 which may be, for example, a prediction of an assigned category 116 to be applied to the unlabeled input.

The above description pertains to a particular kind of AI/ML System 102, which applies supervised learning techniques given available training data with input/result pairs. However, the present invention is not limited to use with a specific AI/ML paradigm, and other types of AI/ML techniques may be used.

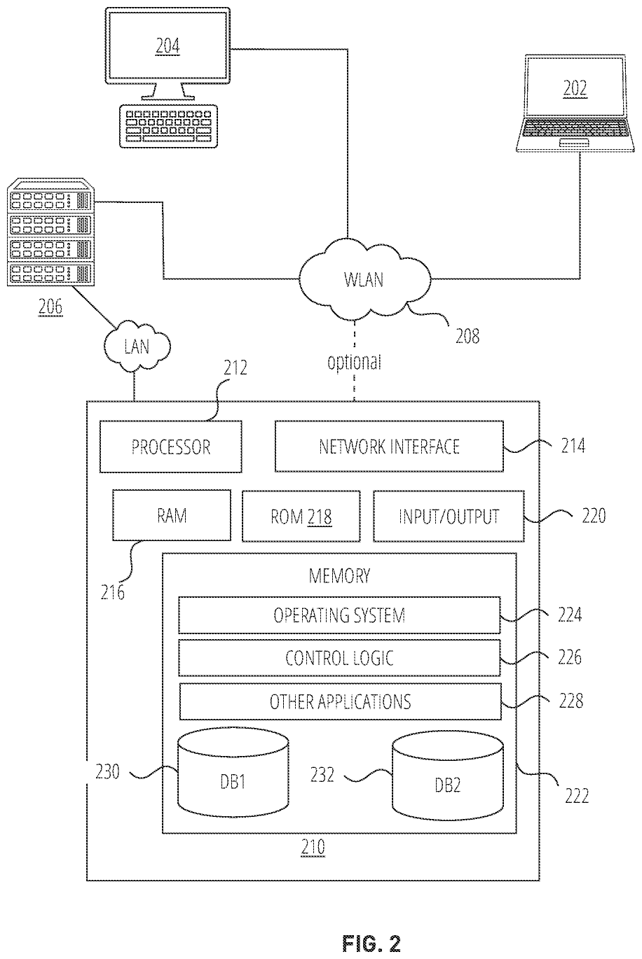

FIG. 2 illustrates one example of a system architecture and data processing device that may be used to implement one or more illustrative aspects described herein in a standalone and/or networked environment. Various network nodes, such as the data server 210, web server 206, computer 204, and laptop 202 may be interconnected via a wide area network 208 (WAN), such as the internet. Other networks may also or alternatively be used, including private intranets, corporate networks, LANs, metropolitan area networks (MANs) wireless networks, personal networks (PANs), and the like. Network 208 is for illustration purposes and may be replaced with fewer or additional computer networks. A local area network (LAN) may have one or more of any known LAN topology and may use one or more of a variety of different protocols, such as ethernet. Devices data server 210, web server 206, computer 204, laptop 202 and other devices (not shown) may be connected to one or more of the networks via twisted pair wires, coaxial cable, fiber optics, radio waves or other communication media.

Computer software, hardware, and networks may be utilized in a variety of different system environments, including standalone, networked, remote-access (aka, remote desktop), virtualized, and/or cloud-based environments, among others.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data—attributable to a single entity—which resides across all physical networks.

The components may include data server 210, web server 206, and client computer 204, laptop 202. Data server 210 provides overall access, control and administration of databases and control software for performing one or more illustrative aspects described herein. Data server 210 may be connected to web server 206 through which users interact with and obtain data as requested. Alternatively, data server 210 may act as a web server itself and be directly connected to the internet. Data server 210 may be connected to web server 206 through the network 208 (e.g., the internet), via direct or indirect connection, or via some other network. Users may interact with the data server 210 using remote computer 204, laptop 202, e.g., using a web browser to connect to the data server 210 via one or more externally exposed web sites hosted by web server 206. Client computer 204, laptop 202 may be used in concert with data server 210 to access data stored therein, or may be used for other purposes. For example, from client computer 204, a user may access web server 206 using an internet browser, as is known in the art, or by executing a software application that communicates with web server 206 and/or data server 210 over a computer network (such as the internet).

Servers and applications may be combined on the same physical machines, and retain separate virtual or logical addresses, or may reside on separate physical machines. FIG. 2 illustrates just one example of a network architecture that may be used, and those of skill in the art will appreciate that the specific network architecture and data processing devices used may vary, and are secondary to the functionality that they provide, as further described herein. For example, services provided by web server 206 and data server 210 may be combined on a single server.

Each component data server 210, web server 206, computer 204, laptop 202 may be any type of known computer, server, or data processing device. Data server 210, e.g., may include a processor 212 controlling overall operation of the data server 210. Data server 210 may further include RAM 216, ROM 218, network interface 214, input/output interfaces 220 (e.g., keyboard, mouse, display, printer, etc.), and memory 222. Input/output interfaces 220 may include a variety of interface units and drives for reading, writing, displaying, and/or printing data or files. Memory 222 may further store operating system software 224 for controlling overall operation of the data server 210, control logic 226 for instructing data server 210 to perform aspects described herein, and other application software 228 providing secondary, support, and/or other functionality which may or may not be used in conjunction with aspects described herein. The control logic may also be referred to herein as the data server software control logic 226. Functionality of the data server software may refer to operations or decisions made automatically based on rules coded into the control logic, made manually by a user providing input into the system, and/or a combination of automatic processing based on user input (e.g., queries, data updates, etc.).

Memory 1122 may also store data used in performance of one or more aspects described herein, including a first database 232 and a second database 230. In some embodiments, the first database may include the second database (e.g., as a separate table, report, etc.). That is, the information can be stored in a single database, or separated into different logical, virtual, or physical databases, depending on system design. Web server 206, computer 204, laptop 202 may have similar or different architecture as described with respect to data server 210. Those of skill in the art will appreciate that the functionality of data server 210 (or web server 206, computer 204, laptop 202) as described herein may be spread across multiple data processing devices, for example, to distribute processing load across multiple computers, to segregate transactions based on geographic location, user access level, quality of service (QoS), etc.

One or more aspects may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium such as a nonvolatile storage device. Any suitable computer readable storage media may be utilized, including hard disks, CD-ROMs, optical storage devices, magnetic storage devices, and/or any combination thereof. In addition, various transmission (non-storage) media representing data or events as described herein may be transferred between a source and a destination in the form of electromagnetic waves traveling through signal-conducting media such as metal wires, optical fibers, and/or wireless transmission media (e.g., air and/or space). various aspects described herein may be embodied as a method, a data processing system, or a computer program product. Therefore, various functionalities may be embodied in whole or in part in software, firmware and/or hardware or hardware equivalents such as integrated circuits, field programmable gate arrays (FPGA), and the like. Particular data structures may be used to more effectively implement one or more aspects described herein, and such data structures are contemplated within the scope of computer executable instructions and computer-usable data described herein.

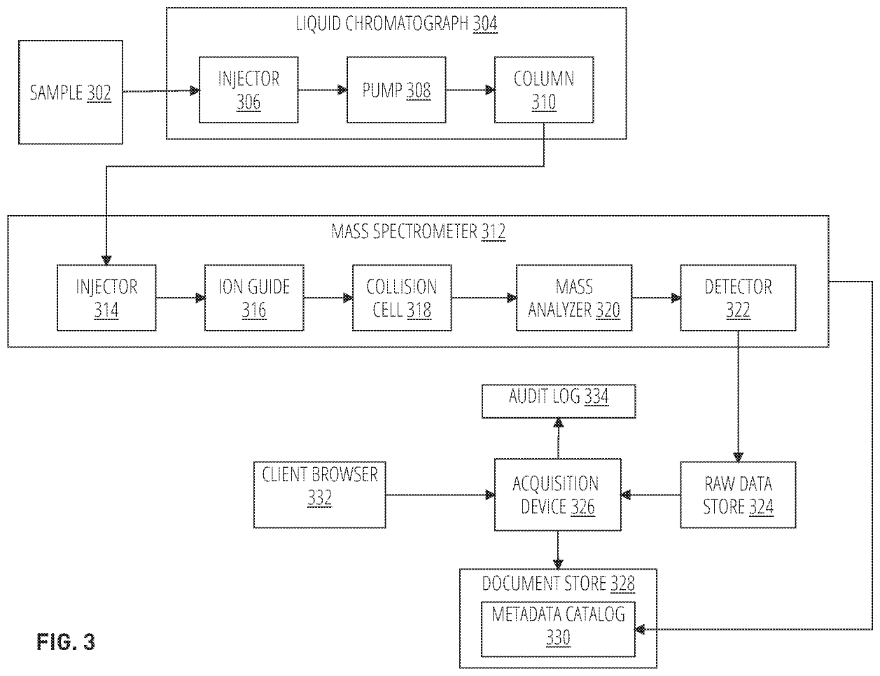

For purposes of illustration, FIG. 3 is a schematic diagram of a system that may be used in connection with techniques herein. Although FIG. 3 depicts particular types of devices in a specific LCMS configuration, one of ordinary skill in the art will understand that different types of chromatographic devices (e.g., MS, tandem MS, etc.) may also be used in connection with the present disclosure.

A sample 302 is injected into a liquid chromatograph 304 through an injector 306. A pump 308 pumps the sample through a column 310 to separate the mixture into component parts according to retention time through the column.

The output from the column is input to a mass spectrometer 312 for analysis. Initially, the sample is desolved and ionized by a desolvation/ionization device 114. Desolvation can be any technique for desolvation, including, for example, a heater, a gas, a heater in combination with a gas or other desolvation technique. Ionization can be by any ionization techniques, including for example, electrospray ionization (ESI), atmospheric pressure chemical ionization (APCI), matrix assisted laser desorption (MALDI) or other ionization technique. Ions resulting from the ionization are fed to a collision cell 318 by a voltage gradient being applied to an ion guide 316. Collision cell 318 can be used to pass the ions (low-energy) or to fragment the ions (high-energy).

Different techniques (including one described in U.S. Pat. No. 6,717,130, to Bateman et al., which is incorporated by reference herein) may be used in which an alternating voltage can be applied across the collision cell 318 to cause fragmentation. Spectra are collected for the precursors at low-energy (no collisions) and fragments at high-energy (results of collisions).

The output of collision cell 318 is input to a mass analyzer 320. Mass analyzer 320 can be any mass analyzer, including quadrupole, time-of-flight (TOF), ion trap, magnetic sector mass analyzers as well as combinations thereof. A detector 322 detects ions emanating from mass analyzer 122. Detector 322 can be integral with mass analyzer 320. For example, in the case of a TOF mass analyzer, detector 322 can be a microchannel plate detector that counts intensity of ions, i.e., counts numbers of ions impinging it.

A raw data store 324 may provide permanent storage for storing the ion counts for analysis. For example, raw data store 324 can be an internal or external computer data storage device such as a disk, flash-based storage, and the like. An acquisition 326 analyzes the stored data. Data can also be analyzed in real time without requiring storage in a storage medium 124. In real time analysis, detector 322 passes data to be analyzed directly to computer 126 without first storing it to permanent storage.

Collision cell 318 performs fragmentation of the precursor ions. Fragmentation can be used to determine the primary sequence of a peptide and subsequently lead to the identity of the originating protein. Collision cell 318 includes a gas such as helium, argon, nitrogen, air, or methane. When a charged precursor interacts with gas atoms, the resulting collisions can fragment the precursor by breaking it up into resulting fragment ions. Such fragmentation can be accomplished as using techniques described in Bateman by switching the voltage in a collision cell between a low voltage state (e.g., low energy, <5 V) which obtains MS spectra of the peptide precursor, with a high voltage state (e.g., high or elevated energy, >15V) which obtains MS spectra of the collisionally induced fragments of the precursors. High and low voltage may be referred to as high and low energy, since a high or low voltage respectively is used to impart kinetic energy to an ion.

Various protocols can be used to determine when and how to switch the voltage for such an MS/MS acquisition. For example, conventional methods trigger the voltage in either a targeted or data dependent mode (data-dependent analysis, DDA). These methods also include a coupled, gas-phase isolation (or pre-selection) of the targeted precursor. The low-energy spectra are obtained and examined by the software in real-time. When a desired mass reaches a specified intensity value in the low-energy spectrum, the voltage in the collision cell is switched to the high-energy state. The high-energy spectra are then obtained for the pre-selected precursor ion. These spectra contain fragments of the precursor peptide seen at low energy. After sufficient high-energy spectra are collected, the data acquisition reverts to low-energy in a continued search for precursor masses of suitable intensities for high-energy collisional analysis.

Different suitable methods may be used with a system as described herein to obtain ion information such as for precursor and product ions in connection with mass spectrometry for an analyzed sample. Although conventional switching techniques can be employed, embodiments may also use techniques described in Bateman which may be characterized as a fragmentation protocol in which the voltage is switched in a simple alternating cycle. This switching is done at a high enough frequency so that multiple high- and multiple low-energy spectra are contained within a single chromatographic peak. Unlike conventional switching protocols, the cycle is independent of the content of the data. Such switching techniques described in Bateman, provide for effectively simultaneous mass analysis of both precursor and product ions. In Bateman, using a high- and low-energy switching protocol may be applied as part of an LC-MS analysis of a single injection of a peptide mixture. In data acquired from the single injection or experimental run, the low-energy spectra contains ions primarily from unfragmented precursors, while the high-energy spectra contain ions primarily from fragmented precursors. For example, a portion of a precursor ion may be fragmented to form product ions, and the precursor and product ions are substantially simultaneously analyzed, either at the same time or, for example, in rapid succession through application of rapidly switching or alternating voltage to a collision cell of an MS module between a low voltage (e.g., generate primarily precursors) and a high or elevated voltage (e.g. generate primarily fragments) to regulate fragmentation. Operation of the MS in accordance with the foregoing techniques of Bateman by rapid succession of alternating between high (or elevated) and low energy may also be referred to herein as the Bateman technique and the high-low protocol.

The data acquired by the high-low protocol allows for the accurate determination of the retention times, mass-to-charge ratios, and intensities of all ions collected in both low- and high-energy modes. In general, different ions are seen in the two different modes, and the spectra acquired in each mode may then be further analyzed separately or in combination. The ions from a common precursor as seen in one or both modes will share the same retention times (and thus have substantially the same scan times) and peak shapes. The high-low protocol allows the meaningful comparison of different characteristics of the ions within a single mode and between modes. This comparison can then be used to group ions seen in both low-energy and high-energy spectra.

In summary, such as when operating the system using the Bateman technique, a sample 302 is injected into the LC/MS system. The LC/MS system produces two sets of spectra, a set of low-energy spectra and a set of high-energy spectra. The set of low-energy spectra contain primarily ions associated with precursors. The set of high-energy spectra contain primarily ions associated with fragments. These spectra are stored in a raw data store 324. After data acquisition, these spectra can be extracted from the raw data store 324 and displayed and processed by post-acquisition algorithms in the acquisition 326.

Metadata describing various parameters related to data acquisition may be generated alongside the raw data. This information may include a configuration of the liquid chromatograph 304 or mass spectrometer 312 (or other chromatography apparatus that acquires the data), which may define a data type. An identifier (e.g., a key) for a codec that is configured to decode the data may also be stored as part of the metadata and/or with the raw data. The metadata may be stored in a metadata catalog 330 in a document store 328.

The acquisition 326 may operate according to a workflow, providing visualizations of data to an analyst at each of the workflow steps and allowing the analyst to generate output data by performing processing specific to the workflow step. The workflow may be generated and retrieved via a client browser 332. As the acquisition 326 performs the steps of the workflow, it may read read raw data from a stream of data located in the raw data store 324. As the acquisition 326 performs the steps of the workflow, it may generate processed data that is stored in a metadata catalog 330 in a document store 328; alternatively or in addition, the processed data may be stored in a different location specified by a user of the acquisition 326. It may also generate audit records that may be stored in an audit log 334.

The exemplary embodiments described herein may be performed at the client browser 332 and acquisition 326, among other locations. An example of a device suitable for use as an acquisition 326 and/or client browser 332, as well as various data storage devices, is depicted in FIG. 2.

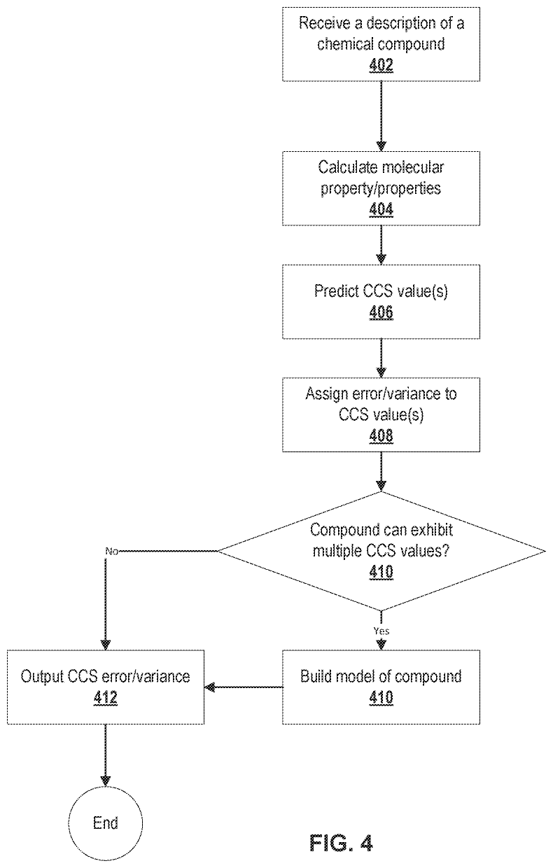

FIG. 4 is a flowchart depicting exemplary logic suitable for practicing exemplary embodiments. The logic depicted in FIG. 4 may be embodied as a method performed by a suitable computing device, a non-transitory computer readable medium storing instructions for performing the logic, and/or a computing apparatus configured to perform the logic (among other possibilities). Although FIG. 4 depicts particular steps or procedures performed in a particular order, one of ordinary skill in the art will recognize that certain steps/procedures may be performed out-of-order or omitted entirely. Furthermore, additional or different steps or procedures may be added to the logic.

At block 402, a computing apparatus may receive a description of a chemical compound. Among other possibilities, the compound may represent a peptide or a small molecule.

At block 404, the computing apparatus may calculate one or more molecular properties of the chemical compound described in block 402. For example, in the case of a peptide, the molecular properties may represent one or more of a length normalized residue value or a Van der Waals volume. In the case of the length normalized residue value specifically, the value may be used to differentiate 3+(and possibly other higher charge state) branches of the compound. In some embodiments, the molecular properties may represent a structural flexibility.

At block 406, one or more collision cross-section (CCS) values may be predicted for the chemical compound. The predicted CCS value(s) may represent an assignment of a charge to a particular location in the chemical compound. In some cases, the charge may exist at different locations on the chemical compound, based on the structure of the compound (for example, at different times).

The CCS value(s) may be predicted using molecular modeling or machine learning. An exemplary process for predicting CSS values using molecular modeling and/or machine learning is described in U.S. Pat. No. 11,226,309, which issued on Jan. 18, 2022 and is entitled “Techniques for Predicting Collision Cross-Section Values.”

At block 408, an error or variance may be assigned to each of the CCS values predicted at block 406. The system may use the molecular properties calculated at block 404 to assign the error/variance. For example, qualitative relationships exist between the structure and observed prediction error. These relationships are related to the location of the charge, such as whether the charge exists at one or more locations, or is distributed over the surface of the molecule. The location where the charge remains the majority of the time may affect the error/variance. Other factors, such as the compound structural flexibility, the Van der Waals radius (for peptides), or “light” molecular modeling (for small molecules) may also factor into the error/variance determination.

When the system assigns a charge to a specific location, this assignment may be associated with a degree of uncertainty (e.g., when the charge can exist at multiple possible locations based on the machine learning process or molecular modeling). This uncertainty may factor into the error or variance. Moreover, when the uncertainty or error/variance exceeds a certain predetermined threshold value (which may be a default value or may be user-configurable), this situation may be flagged in the output generated at block 414.

At block 410, the computing apparatus may determine whether the compound is capable of exhibiting more than one CCS value. The different CCS values may represent, for example, a plurality of possible locations for a charge on the compound.

If the determination at block 410 is “no,” then processing may proceed to block 414 and the error/variance assigned at block 408 may be output. For example, the error/variance may be displayed in a GUI on a suitable display device, or may be provided to other procedures or algorithms to perform computations based on the error or variance value.

If the determination at block 410 is “yes,” then processing may proceed to block 412 where the computing apparatus builds a model of the compound. When building the model, the plurality of different possible locations for the charge on the compound may be captured and represented in the model.

For instance, some embodiments may determine that a given compound is likely to be associated with more than one CCS value, or a CCS prediction value with more variation associated with it (since the structure cannot accommodate the charge). If a compound can exhibit more than one CCS value, since the structure can accommodate a charge at more than one location, this information could be captured/encoded when the model is created, thereby providing the ability to report more than one CCS value. Or, additionally, when charge assignment is challenged, this may be flagged and reported in the output when CCS values are computed (in both ML and MM).

Processing may then proceed to block 410, where the errors or variances may be output.

### Experimental Example

In one example provided here for purposes of illustration of the concept, twenty small molecules were randomly selected from a library for detailed analysis. Five conformers using two models (root mean square deviation (RMSD) diversity or energy optimized) were created for each set of 20 structures (20 compounds with CCS prediction errors residing in the >90% percentile range (more than) and 20 compounds with CCS prediction errors residing in the <100% percentile range) using openBabel (obabel).

Next, with openBabel (obgen), each conformer was (first pass) energy minimized.

The resultant 3D structure files were converted to pdb files and CCS values determined with the Windows version of MobCal, proving Exact hard-spheres scattering (EHS) and projection approximation (PA) calculated CCS values.

For each compound and model, a mean and standard deviation was calculated.

Standard deviation/mean values (measure for structural flexibility) frequency distributions were calculated for the two groups and two models.

The machine learning predicted and Molcal calculated CCS values vs. the experimental CCS measurement results were determined for the protonated (top) and deprotonated (bottom) molecules, and suggested that the error is related with structure (and method independent).

Based on the above, the chemical diversity and characteristics of the molecules in solution at pH 2 and 12 (ionization mode dependent) were examined by manually and computationally attempting a charge to the molecule.

Isoelectric point (pI)/pKA calculations were performed for the so-called LessThan group (where there was good agreement between predicted and experimental CCS values). The results suggested unambiguous charge assignment in solution at pH 2.

The equivalents for the MoreThan group (where there was relatively poor agreement between predicted and experimental CCS values) were then calculated, which suggested more ambiguous assignment of a charge in solution at pH 2 (charges ranged from 0 to 0.42).

Subsequently all compounds (20 (LessThan) vs. 20 (MoreThan)) were considered. Charge assignment was manually classified, suggesting some separation of groups—mostly the group where the charge could be unambiguously assigned to a structure/molecule (both manually and computationally). Separation was less obvious when various pKa metrics were used to provide a results summary. Note that at this stage of the analysis, protonated and deprotonated structures were not analyzed separately.

Chemical compounds that are known to form conformers, and as such could be seen as compounds with ambiguous charge assignment, were processed similar as described above.

The results suggest that these molecules (Q) group more closely together with the compounds that have a larger prediction error associated with them (MoreThan group).

A number of compounds were selected for Gaussian and processing (compounds that had a median mass and were on/off the structural flexibility—standard deviation/median calculated CCS—vs. m/z trend line).

Neutral and charged versions of the conformers were created. Interestingly, openBabel automatically assigned a charge to the structures during creation of the conformers of the group of compounds that illustrated good agreement between prediction and experimental CCS values (LessThan). Hence, the charge (proton) was removed to create neutral equivalents. For the other group of compounds, illustrating poor agreement between prediction and measurement (MoreThan), a tool was used to computationally assign a charge to the most likely position. In these cases, openBabel created a neutral version of the conformer structures.

Software was used to determine proton affinity and zero-point energy values. Intermediate calculated results were used to illustrate differences between the two groups of structures. The results show the total energy (left) and Coulomb energy (right) for all compounds and ionization modes combined. Normalized total and Coulomb energy values were determined (energy values divided by the molecular weight equivalents).

Next, proton affinity values were calculated using the minimal energy values for the charged and neutral species for a given conformer set.

Next, the computational results of the solution and gas phase calculation were contrasted. Shown left summarizes the overall pKa based calculations for the two structure groups. Correlation appeared to be present but may be ‘cluttered’ as different charge types (protonated/deprotonated) are combined.

The final comparison only considered the protonated species and excluding ‘outliers’, showing from left to right ΔpKa (highest minus lowest), protein affinity, and ΔpKa vs. proton affinity.

Proton affinity calculations were confirmed with higher level theory methods (DFT-Gaussian). The calculated proton affinity values using ADF and DFT correlated, and a better correlation was obtained for the ‘LessThan’ group, i.e. more ambiguous results were obtained for the ‘MoreThan’ group. Equivalents to the summary distributions were determined for proton affinity and ΔpKa vs. proton affinity using DFT calculated proton affinity values. The same data was calculated with one of the compounds removed to illustrate that the obtained results using DFT and ADF theory demonstrate similar trends.

In conclusion, generally, the MoreThan structures are more ambiguous in predictions results, within group consistency molecular modeling, charge assignment and separate themselves based on these ‘properties’ from the ‘LessThan’ set of molecules.

### CONCLUSION

The components and features of the devices described above may be implemented using any combination of discrete circuitry, application specific integrated circuits (ASICs), logic gates and/or single chip architectures. Further, the features of the devices may be implemented using microcontrollers, programmable logic arrays and/or microprocessors or any combination of the foregoing where suitably appropriate. It is noted that hardware, firmware and/or software elements may be collectively or individually referred to herein as “logic” or “circuit.”

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

At least one computer-readable storage medium may include instructions that, when executed, cause a system to perform any of the computer-implemented methods described herein.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

A procedure is here, and generally, conceived to be a self-consistent sequence of operations leading to a desired result. These operations are those requiring physical manipulations of physical quantities. Usually, though not necessarily, these quantities take the form of electrical, magnetic or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, the manipulations performed are often referred to in terms, such as adding or comparing, which are commonly associated with mental operations performed by a human operator. No such capability of a human operator is necessary, or desirable in most cases, in any of the operations described herein, which form part of one or more embodiments. Rather, the operations are machine operations. Useful machines for performing operations of various embodiments include general purpose digital computers or similar devices.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Various embodiments also relate to apparatus or systems for performing these operations. This apparatus may be specially constructed for the required purpose or it may comprise a general purpose computer as selectively activated or reconfigured by a computer program stored in the computer. The procedures presented herein are not inherently related to a particular computer or other apparatus. Various general purpose machines may be used with programs written in accordance with the teachings herein, or it may prove convenient to construct more specialized apparatus to perform the required method steps. The required structure for a variety of these machines will appear from the description given.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

What is claimed is:

1. A computer-implemented method comprising:
receiving a description of a chemical compound;
calculating one or more molecular properties for the chemical compound;
predicting a collision cross-section (CCS) value for the chemical compound; and
using the one or more calculated molecular properties to assign an error or variance to the predicted CCS value.

2. The method of claim 1, wherein the CCS value is an assignment of a charge to a particular location in the chemical compound.

3. The method of claim 1, wherein the compound is a peptide and the one or more molecular properties comprises one or more of a length normalized residue value or a Van der Waals volume.

4. The method of claim 1, wherein the chemical compound is a small molecule.

5. The method of claim 1, wherein the molecular properties comprise a structural flexibility.

6. The method of claim 1, further comprising:
determining that the compound is capable of exhibiting a plurality of CCS values indicating a plurality of possible locations for a charge on the compound; and
building a model of the chemical compound, the building comprising capturing the plurality of locations for the charge.

7. The method of claim 1, wherein the CCS value is predicted using one or more of molecular modeling or machine learning.

8. A non-transitory computer-readable medium storing instructions that, when executed by a processor, cause the processor to
receive a description of a chemical compound;
calculate one or more molecular properties for the chemical compound;
predict a collision cross-section (CCS) value for the chemical compound; and
use the one or more calculated molecular properties to assign an error or variance to the predicted CCS value.

9. The medium of claim 8, wherein the CCS value is an assignment of a charge to a particular location in the chemical compound.

10. The method of claim 1, wherein the compound is a peptide and the one or more molecular properties comprises one or more of a length normalized residue value or a Van der Waals volume.

11. The medium of claim 8, wherein the chemical compound is a small molecule.

12. The medium of claim 8, wherein the molecular properties comprise a structural flexibility.

13. The medium of claim 8, further storing instructions for:
determining that the compound is capable of exhibiting a plurality of CCS values indicating a plurality of possible locations for a charge on the compound; and
building a model of the chemical compound, the building comprising capturing the plurality of locations for the charge.

14. The medium of claim 8, wherein the CCS value is predicted using one or more of molecular modeling or machine learning.

15. An apparatus comprising:
a hardware processor and
a non-transitory computer-readable medium storing instructions that, when executed by the processor, cause the processor to:
receive a description of a chemical compound;
calculate one or more molecular properties for the chemical compound;
predict a collision cross-section (CCS) value for the chemical compound; and
use the one or more calculated molecular properties to assign an error or variance to the predicted CCS value.

16. The apparatus of claim 15, wherein the CCS value is an assignment of a charge to a particular location in the chemical compound.

17. The apparatus of claim 15, wherein the compound is a peptide and the one or more molecular properties comprises one or more of a length normalized residue value or a Van der Waals volume.

18. The apparatus of claim 15, wherein the molecular properties comprise a structural flexibility.

19. The apparatus of claim 15, wherein the medium further stores instructions for:
determining that the compound is capable of exhibiting a plurality of CCS values indicating a plurality of possible locations for a charge on the compound; and
building a model of the chemical compound, the building comprising capturing the plurality of locations for the charge.

20. The apparatus of claim 15, wherein the CCS value is predicted using one or more of molecular modeling or machine learning.
